(19) United States
(12) Patent Application Publication
(10) Pub. No.: US 2009/0105538 A1
(43) Pub. Date: Apr. 23, 2009

(54) Endoscope System

(76) Inventors: Jacques Van Dam, San Carlos, CA (US); Lex Bayer, Palo Alto, CA (US)

Correspondence Address: SQUIRE, SANDERS & DEMPSEY L.L.P., 1 MARITIME PLAZA, SUITE 300, SAN FRANCISCO, CA, 94111, US

(21) Appl. No.: 12/181,280

(22) Filed: Jul. 28, 2008

Related U.S. Application Data
(60) Provisional application No. 60/952,204, filed on Jul. 26, 2007.

(51) Int. Cl. A61B 1/04; G06F 3/041; H04N 5/38

(52) U.S. Cl. 600/109; 345/173; 348/65; 348/E05.093

(57) ABSTRACT

An endoscope system includes a catheter having a camera module, a wall mounted unit including an LCD screen, and a control box that processes video images captured by the camera module and output video signals to the LCD screen to display the captured video images.

This application claims the benefit of U.S. Provisional Patent Application No. 60/952,204, filed Jul. 26, 2007, the entire disclosure of which is incorporated herein by reference.

### TECHNICAL FIELD OF THE INVENTION

The present invention relates to an endoscope, in particular to a gastroscope. The present invention relates also to a method for detecting Barrett's esophagus.

### BACKGROUND OF THE INVENTION

An endoscope is a medical device comprising a flexible tube and a camera mounted on the distal end of the tube. The endoscope is insertable into an internal body cavity through a body orifice or a surgical incision to examine the body cavity and tissues for diagnosis. The tube of the endoscope has one or more longitudinal channels, through which an instrument can reach the body cavity to take samples of suspicious tissues or to perform other surgical procedures such as polypectomy.

There are many types of endoscopes, and they are named in relation to the organs or areas with which they are used. For example, gastroscopes or esophagoscopes are used for examination and treatment of the esophagus, stomach and duodenum; colonoscopes for the colon; bronchoscopes for the bronchi; laparoscopes for the peritoneal cavity; sigmoidoscopes for the rectum and the sigmoid colon; arthroscopes for joints; cystoscopes for the urinary bladder; and angioscopes for the examination of blood vessels.

Current endoscopes require an array of equipment, which provide control and power to the camera and a light source for the camera, and process and display video signals from the camera. Due to the necessary ancillary equipment, current endoscopes' portability is limited, and they are difficult to use. The expense and complexity of the equipment and procedure prohibit the use of endoscopes outside of hospitals, Ambulatory Surgery Centers, and some gastrointestinal specialists' offices. And screening for certain diseases such as Barrett's esophagus is performed for only a small percentage of patients, for whom such a procedure would be beneficial. A smaller and less expensive endoscope would allow for more widespread use in the medical industry and potentially reduce the mortality associated with certain diseases.

Accordingly, there exists a need for a compact and operator-friendly endoscope such as a gastroscope. Such a gastroscope can be employed by primary care physicians and other non-specialists.

### SUMMARY OF THE INVENTION

According to one aspect of the invention, an endoscope system includes a catheter having a camera module, a wall mounted unit including an LCD screen, and a control box that processes video images captured by the camera module and output video signals to the LCD screen to display the captured video images.

According to one embodiment of the invention, the system further includes a plurality of catheters.

According to another embodiment of the invention, the lengths of the catheters vary.

According to still another embodiment of the invention, the stiffness levels of the catheters vary.

According to yet another embodiment of the invention, the catheters are single-use catheters.

According to yet still another embodiment of the invention, each catheter includes a camera module.

According to a further embodiment of the invention, one of the camera modules is a disposable camera module designed for examining a patient's ear and another of the camera modules is a disposable camera module designed for examining a patient's nasal cavities.

According to a still further embodiment of the invention, the image sensor sizes and optical characteristics of the camera modules vary.

According to a yet further embodiment of the invention, each catheter has a proximal end and a distal end, and has a connector at the proximal end.

According to a yet still further embodiment of the invention, the connector has electrical contacts for relaying electrical and communication signals.

According to another embodiment of the invention, the camera module includes an LED and a light pipe for transmitting light generated by the LED.

According to still another embodiment of the invention, the wall mounted unit includes a handle that is detachably connectable to the catheter.

According to yet another embodiment of the invention, the wall mounted unit further includes a back panel, an interface module, an air pump that sends air to the handle.

According to yet still another embodiment of the invention, the LCD screen is a touch sensitive display having software controlled buttons, whereby an operator is able to perform control functions by touching the buttons.

According to another aspect of the invention, a method of detecting Barrett's esophagus includes inserting a catheter of a gastroscope system into a patient's esophagus; identifying an area of known esophageal tissue on a screen of the gastroscope system, and setting a first base line point in terms of image properties in the area of esophageal tissue; identifying an area of known stomach epithelial tissue on the screen of the gastroscope system, and setting a second base line point in terms of image properties in the area of stomach epithelial tissue; identifying areas of stomach epithelial cells on the screen based on the first and second base points; and accentuating the identified areas of stomach epithelial cells.

According to a further embodiment of the invention, the step of identifying the areas of stomach epithelial cells includes analyzing the areas for various color properties.

According to another embodiment of the invention, the method further includes measuring the degree of metaplasia by analyzing color properties.

According to yet another aspect of the invention, a method of detecting Barrett's esophagus includes inserting a catheter of a gastroscope system into a patient's esophagus; identifying an area of known esophageal tissue on a screen of the gastroscope system, and setting a base line point in terms of image properties in the area of esophageal tissue; identifying areas of stomach epithelial cells on the screen based on the base points; and accentuating the identified areas of stomach epithelial cells.

According to still another aspect of the invention, a method of detecting Barrett's esophagus includes inserting a catheter of a gastroscope system into a patient's esophagus; identifying an area of known stomach epithelial tissue on the screen of the gastroscope system, and setting a base line point in terms of image properties in the area of stomach epithelial tissue; identifying areas of stomach epithelial cells on the screen based on the base points; and accentuating the identified areas of stomach epithelial cells.

According to a further aspect of the invention, a method for determining a length of metaplasia includes inserting a catheter of a gastroscope system into a patient's esophagus; identifying upper and lower borders of the area of metaplasia; moving a camera module of the gastroscope system from one of the upper and lower borders to the other while capturing partial images of the interior surface of the esophagus; identifying similar regions or corresponding key points between two captured images; calculating a distance by which a key point or corresponding area has moved from the earlier one of the two images to the later of the two images; and obtaining a length of metaplasia by adding the calculated distances.

### BRIEF DESCRIPTION OF DRAWINGS

FIG. 1 shows a gastroscope system of the present invention.

FIG. 2 shows a catheter of the gastroscope system shown in FIG. 1.

FIG. 3 shows a cut away view of the catheter of FIG. 2.

FIG. 4 shows a front exploded view of the camera module of the catheter of FIG. 2.

FIG. 5 shows a side exploded view of the camera module of FIG. 4.

FIG. 6 shows a perspective view of the camera module of FIG. 4.

FIG. 7 shows a perspective view of a wall mount unit of the gastroscope system shown in FIG. 1.

FIG. 8 shows a rear exploded view of a pump/interface housing of the wall mount unit of FIG. 7.

FIG. 9 shows a side view of a handle of the catheter of FIG. 2.

FIG. 10 shows a perspective view of a control box of the gastroscope system shown in FIG. 1.

### DESCRIPTION OF PREFERRED EMBODIMENTS

The preferred embodiments described below are “gastroscopes,” which are endoscopes employed to view the upper gastrointestinal tract. While gastroscopes are described as preferred embodiments, it will be obvious to those skilled in the art that the features of the gastroscopes are equally applicable to any endoscopes and should not be limited to gastroscopes. The present invention, therefore, is not limited to gastroscopes. The appended claims define the scope of the present invention.

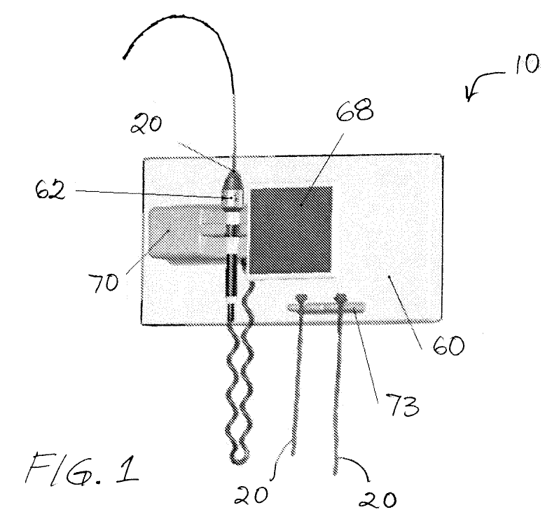

FIGS. 1 and 10 illustrate a gastroscope system 10 (FIG. 1) that may be divided into 3 main components: one or more catheters 20 (FIG. 1), one or more of which are preferably disposable; a wall mount unit 60 (FIG. 1) which is preferably reusable; and a control box 90 (FIG. 10) which is preferably reusable.

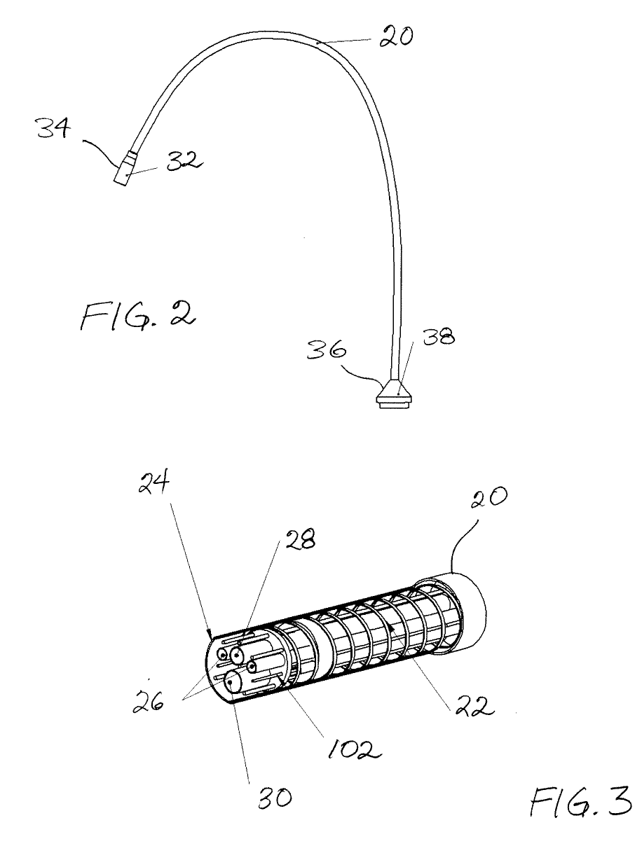

FIG. 2 provides a more detailed view of the catheter 20. The catheter 20 preferably is constructed from a material that is both flexible and rigid enough such that it can be pushed through the patient's upper gastrointestinal tract. For example, the catheter 20 may be made of a plastic that is biocompatible. As shown in FIG. 3, the catheter 20 may include an underlying braided coil 22 and a flexible sheath covering 24. The catheter 20 may include one or more lumens 26, 28, 30, and a plurality of electrical wires may extend through one or more of the lumens 26 to carry communications and electrical signals between the wall mount unit 60 and a camera module 32 of the catheter 20. In addition, one of the lumens 28 may carry air from a handle 62 (FIG. 1) to the distal end 34 of the catheter 20. The camera module 32 and handle 62 will be described in detail below. In a preferred embodiment, either or both of the camera module 32 and handle 62 may have a diameter that is greater than the rest of the catheter 20. For example, the diameter of the rest of the catheter 20 may 90%, 80%, 70%, 60%, 50%, 40% or 30% of the diameter(s) of the camera module 32 and/or handle 62. The air lumen 28 may be made from a plastic such as PTFE or rubber such as silicone. On the proximal end 36, the catheter 20 has a connector 38, preferably made from a rigid plastic, that can be detachably connected to a complimentary connector 64 on the distal tip 66 of the handle 62. Each of the connectors 38 and 64 may include a plurality of metal contacts (not shown) in order to relay electrical and communication signals. The catheter 20 may include a fluidic connector (not shown) in order to transport air.

As shown in FIGS. 4-6, the camera module 32 may include a printed circuit board (PCB) 40, a light emitting diode (LED) 42 that provides illumination for the camera module 32, a light pipe 44 for transmitting of the light generated by the LED 42, a bezel 46, a lens assembly 48, an image sensor 50, and a camera housing 52. In this preferred embodiment, the camera module 32 communicates with the wall mounted unit 60 through wires that run through a lumen 30 of the catheter 20. These wires also provide power to the camera module 32.

The light pipe 44 (which is preferably translucent), bezel 46, and camera housing 52 are preferably fabricated from a biocompatible plastic such as polypropylene. The methods of joining the light pipe 44, bezel 46, and camera housing 52 include, for example, snap fit, adhesives, and screw fasteners. The lens assembly 48 and image sensor 50 are joined together and then placed against the distal end of the PCB 40 as illustrated in FIG. 5. The LED is secured to the PCB by means of adhesive bonding. The image sensor 50 is preferably an electronic device which converts light incident on photosensitive semiconductor elements into electrical signals. The signals from the sensor 50 are digitized and used to reproduce the image that was incident on the sensor 50. Two commonly used types of image sensors are Charge Coupled Devices (CCD) and Complementary Metal Oxide Semiconductor (CMOS) camera chips.

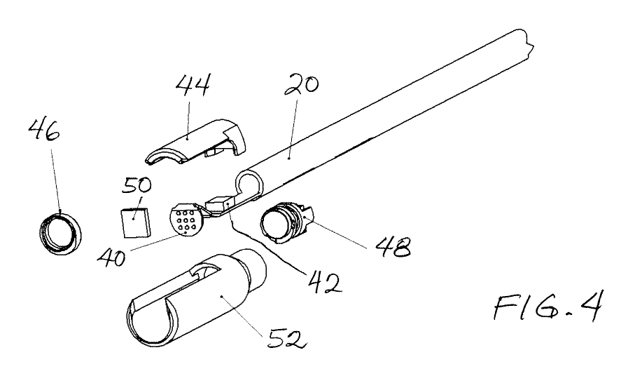

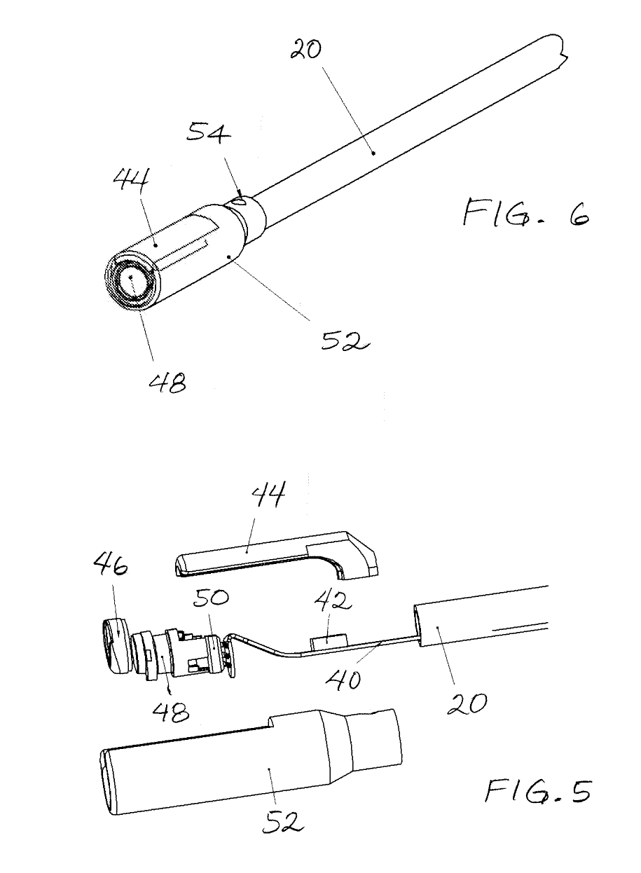

After the inner components have been joined, the outer components are fastened together to sealingly form the camera module 32. The seal preferably is water tight so any moisture from the medical procedure does not enter the camera module 32. The seal may be formed by ultrasonic welding or adhesive bonding. The camera module 32 may also include a hole 54 (FIG. 6) in order to allow the passage of air from the insufflation lumen 28 into the cavity. The methods of securing the camera module 32 to the catheter 20 include heat shrinking and adhesive bonding.

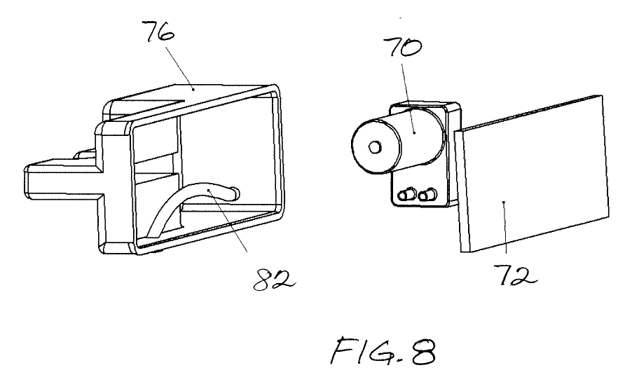

As shown in FIGS. 1, 7 and 8, the wall mount unit 60 preferably includes the handle 62 that can be connected to the catheter 20, an LCD screen 68, a pump 70 for air insufflation, and an interface module 72 having a PCB, a catheter holder 73, and a back panel 74.

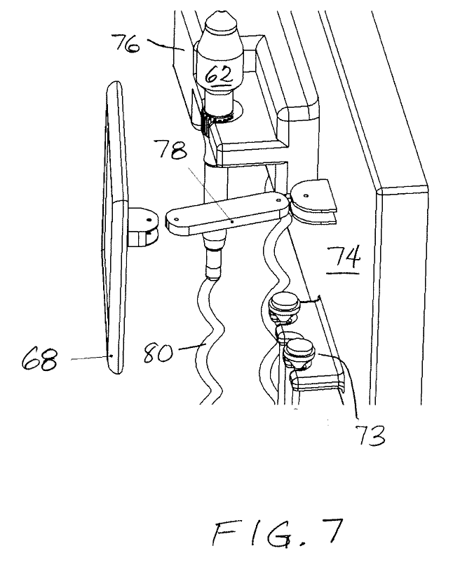

The pump 70 may send air through the handle 62 and the distal end 34 of the catheter 20. The air pump 70 and interface module 72 are placed inside a pump/interface housing 76 and attached to the back panel 74 as shown in FIG. 7. They can be attached to the back panel 74 by means of fasteners or adhesive bonding. The catheter holder 73, which is used to hold catheters 20 when the catheters 20 are detached from the handle 62, may also be attached to the back panel 74.

In the preferred embodiment, the LCD screen 68 is a touch sensitive display so that the operator can control the gastroscope system 10 by touching software controlled buttons on the screen 68. Using the touch-screen LCD, the operator can vary brightness and other settings, and can obtain still images by pressing a button on the touch-screen. In this manner, the operator can perform gastroscopic procedures in an efficient and inexpensive manner. The LCD screen 68 may be attached through an arm mechanism 78 as shown in FIG. 7. Alternately, an arm mechanism such as a VESA mount can be purchased off the shelf and bolted to the back of the LCD screen 68 and the back panel 74.

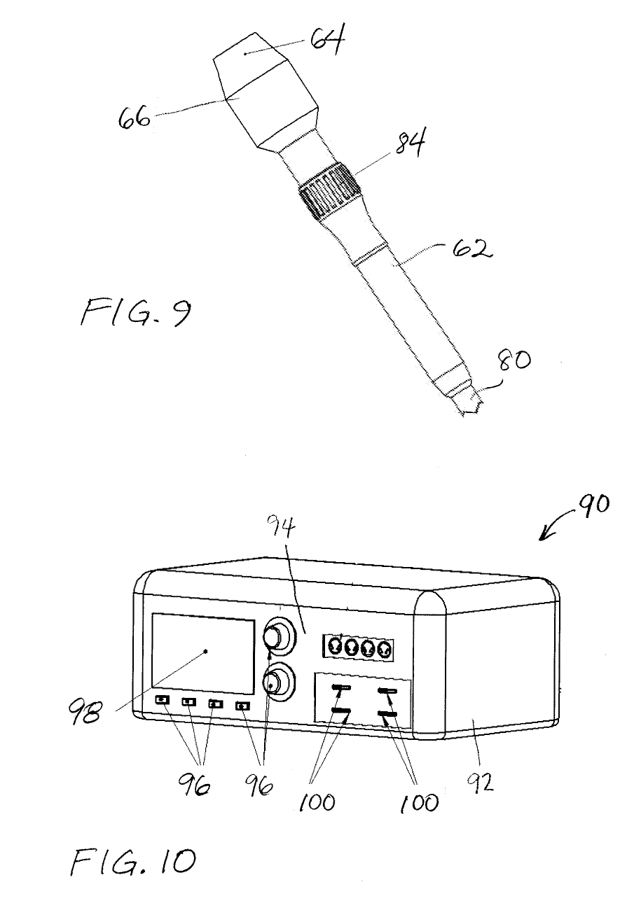

In the illustrated embodiment, the handle 62 is connected to the air pump 70 and the interface module 72 at the proximal end through a single cable 80 that includes a fluidic tube 82 and a plurality of wires. The handle 62 may be a molded or machined piece that is constructed from a plastic or metal. The handle 62 preferably is designed to be ergonomic and allows the operator to transmit a torque to the catheter's distal tip 34 by employing a grooved feature 84 as illustrated in FIG. 9. As stated previously, the handle 62 includes an electric/fluidic connector 64 at its distal tip 66 which mates with the connector 38 of the catheter 20. In the preferred embodiment, the connector 64 of the handle 62 includes a plurality of electrical contacts which transmit electrical and communication signals and one fluidic channel which transports air through the handle 62 to the distal tip 34 of the catheter 20.

The control box 90 includes circuitry and computer hardware for processing video images captured by the camera module 32 and outputting video signals to the LCD screen 68 to display the captured video images. As illustrated in FIG. 10, the control box 90 may include a chassis 92 that has a front panel 94 with control buttons 96. In the preferred embodiment, it includes a digital screen 98 to display information and various connectors 100 for syncing with the wall mount unit 60 and additional monitors/LCDs (not shown). The control box 90 in the preferred embodiment includes computer hardware along with a video capture board that interfaces with the interface module 72 of the wall mount unit 60. In the preferred embodiment, there is a combined cable that includes power and video in order to connect to the wall mount unit 60, while a second cable allows for communication through a serial protocol with the wall mount unit 60.

After the interface module 72 of the wall mount unit 60 receives signals from the camera module 32, the signals are amplified and relayed to the control box 90 for processing. The video capture card of the control box 90 processes the video signal in order to enhance image quality, extracts still images, and converts the video format to other output formats. Once the video images have been processed, they are sent to the LCD screen 68 of the wall mount unit 60 via the control box's graphics card for display. The various image sensor output formats and video signal processing integrated circuits are well documented and understood in the consumer electronics industry and so this process is not explained in further detail.

After the above procedure is completed, video or still images can be transferred to a personal computer (not shown) from the control box 90 by either removing the memory card or transferring the images via the serial interface. Due to the existence of electronic medical records (EMR) at certain medical facilities, still and video images from the procedure can be recorded in a patient's EMR file. The image processing capabilities of the control box 90 can convert the image and video data to a compatible format such as .jpg, mpg, or others for filing in the patient's EMR. In addition, data can be retained in the control box 90 for a period of time by assigning a unique identifier to the corresponding images of each procedure. Video and still images can also be employed in telemedicine applications. After the data has been uploaded into the computer, it can be electronically sent to anyone with a personal computer. Hence, it would be possible for a non-specialist such as a general practitioner to perform the procedure and then transmit the video or still images to a specialist for analysis.

The control box 90 preferably includes algorithms to aid in the detection of Barrett's esophagus. Barrett's esophagus is a metaplasia of the esophageal epithelial tissue near the pyloric sphincter. The smooth, unique lining of the esophagus begins to mimic the structure of the stomach's epithelial layer. The degree of metaplasia is measured by the height of the section above the pyloric sphincter that has started to mimic stomach tissue, and the height of the section is also the basis for diagnosis. In order to facilitate the identification of Barrett's esophagus, the software interface can accentuate areas where there are epithelial cells of stomach origin on the LCD screen 68. By employing an operator interface through the LCD screen 68, the program allows the operator to set base levels. First, the operator may identify an area of tissue that is clearly esophageal in origin. Next, the operator may set a second base point near the pyloric sphincter in an area which clearly has stomach epithelial tissue. Given these two base lines, as the doctor is visualizing the esophagus, the software can then highlight areas on the LCD screen 68 in real-time that are likely to be more similar to epithelial cells of stomach origin and hence potentially Barrett's disease. The algorithm can identify epithelial cells of stomach origin and measure the degree of metaplasia by analyzing the images for various properties, such as hue and other color parameters.

An additional feature of the algorithm is the ability to measure the length or amount of metaplasia. In order to accomplish this task, the algorithm can ascertain the camera tracking distance in a manner similar to an optical computer mouse. In order to accomplish this task, the algorithm analyzes the distance feature points or corresponding areas in each image have moved relative to the previous image. The distance by which a given point or feature moves is denoted by the number of image pixels. Each pixel is then standardized to an actual measurement in units of distance such that the calculation can be performed. The system can automatically find the length of the metaplasia by first identifying areas of metaplasia and then measuring the length of the given segment of metaplasia by looking for upper and lower borders where the metaplasia becomes normal, esophageal tissue. U.S. patent application Ser. No. 12/101,050, which is incorporated herein by reference, describes a similar approach.

In general, the operator may set a baseline level in a region of the esophagus by, for example, pressing a button to instruct the control box to calibrate based on one or more factors, such as the color of the tissue. The control box can then emphasize regions that are dissimilar to the calibrated tissue.

Alternatively, the software employs feature recognition algorithms to identify the open lumen of the esophagus. This opening is then used as a reference scale for size since it can be correlated with average population size distributions. The length of metaplasia visible in the image is then calculated based on its size relative to the lumen opening.

In a preferred embodiment, either or both of the wall mount unit and control box may be portable. For example, either or both of the wall mount unit and control box can be designed so that either or both can be placed on a cart for transportation.

In one alternative embodiment of the present invention, the camera module 32 communicates with the wall mount unit 60 wirelessly. The circuitry in the camera module 32 and the wall mount unit 60 would both include a wireless transceiver. The camera module 32 would be powered by an integrated battery and would be turned on by a simple switch on the camera module 32. The catheter 20 in such an embodiment need not include any electrical wires for transmitting signals and power between the camera module 32 and the handle 62. In addition, the connector 38 at the proximal end of the catheter 20 and connector 64 at the distal tip of the handle 62 would not need to have metal contacts. U.S. patent application Ser. No. 11/609,838, which is incorporated herein by reference, describes a wireless camera module.

In another alternative embodiment, the distal tip of the gastroscope is steerable. However, the most preferred embodiment is a gastroscoe/espphagasacope that is made of a flexible material discussed in the this specification and that does not have any steering and lumens (working channels). In order to make the distal tip steerable, a predetermined length of the distal tip of the catheter is made relatively more flexible and steering wires 102 are attached at peripheral locations on the distal end 34 of the catheter 20, as illustrated in FIG. 3. These wires 102 are enclosed in Bowden type cables along the length of the catheter 20. Bowden type cables are cables containing a free to move wire contained by a flexible overlying hollow tube. These cables are used to transmit pull-forces and are commonly used in bicycle and motor bike brakes. The steering wires contained in the Bowden cables are attached to controls in the handle 62. Using the controls, the steering wires can be pulled and in turn the distal end will bend in a given direction. A plurality of such wires 102 enclosed in Bowden type cables are used to articulate the distal end in different directions. This embodiment allows the operator to maneuver the catheter 20 to image the upper GI tract. In another embodiment, the controls for the steering are electronic. The steering is actuated by motors which are controlled by buttons.

In yet another alternative embodiment, the gastroscope system 10 (FIG. 1) includes various types of catheters or camera modules. These catheters may vary in length or stiffness. Since patients' anatomies vary, this embodiment allows the customization of procedures to particular patients. Different types of catheters could also be used to image different parts of the body. For example, a disposable camera module designed for examining a patient's ear and a disposable camera module designed for examining a patient's nasal cavities could be connected to the same wall mount handle. These additional imaging devices could vary in terms of image sensor size and resolution, optical characteristics, mechanical shape and form, but would all employ the same standard electrical interface connector for power and communication with the control box.

In yet still another embodiment, the catheter includes an accessory lumen to allow the insertion of instruments to perform a biopsy or other minor procedure. The accessory lumen could also be employed to pass air or water into the body cavity. The catheter with an accessory lumen could be used interchangeably with a regular catheter as they both would fit into the handle. This embodiment is formed by housing a plurality of tubes within a larger catheter as shown in FIG. 3. One of these lumens is large enough for the insertion of instruments. The larger catheter has an outer sheath with underlying braided coil in order to provide flexibility to the entire catheter.

In a further embodiment, the catheter is constructed from a soft plastic such as silicon. An external device such as a guidewire or stylet is used to track the catheter through the patient's upper GI system. In another embodiment, the distal tip of the catheter retains a pre-shaped form. An external stylet and guidewire can be employed to straighten the tip during navigation.

In a still further embodiment, the catheter is not a separate part from the handle. Such an embodiment would require sterilization after each procedure or would be limited to a single use. In another embodiment, only the camera module is replaceable while the handle and catheter are reusable. In an alternate embodiment, the catheter is replaceable while the handle and camera module are reusable.

In a yet further embodiment, the handle can be designed in a number of shapes and forms. The handle can also vary in shape depending on the body part that is being imaged.

In another embodiment, the catheter employs fiber optics and a non-digital camera module to transfer images to the handle. The fiber optics may be disposed in a lumen of the catheter. The plurality of fiber optic cables would be secured as a bundle in the lumen to ensure the flexibility of the cable. The camera module at the distal end of the catheter captures the images and transmits the images by bouncing light signals within the fiber optic cables. The control box receives the light signals and digitizes them for display on the LCD screen or other output.

In still another embodiment, the camera module at the distal end of the catheter is incorporated with features such as digital zoom and digital image stabilization. Digital zoom and image stabilization are features that can be incorporated into the image processing IC in the interface board of the wall mount unit. Digital zoom electronically magnifies the image, which is compromised of many pixels. Digital image stabilization analyzes each frame of video for shifts of image pixels and then correcting for these movements.

In an alternate embodiment, the circuitry of the control box such as a video capture card, video graphics card, computer hardware such as a CPU, hard drive, RAM, serial interface, and power supply are incorporated into the wall mount unit. All controls also are on the wall mount unit or are accessible through a touch screen interface on the LCD screen.

In another embodiment, the control box or wall mount unit can be connected to a printer. In such a setup, the operator will be able to print images taken by the camera module. In addition, the control box or wall mount unit can also be configured with an ethernet card in order to allow internet access. Such an embodiment can be used in telemedicine or for incorporating images and videos into EMR.

1. An endoscope system comprising:
a catheter having a camera module;
a wall mounted unit including an LCD screen; and
a control box that processes video images captured by the camera module and output video signals to the LCD screen to display the captured video images.

2. The endoscope system of claim 1, further comprising a plurality of catheters.

3. The endoscope system of claim 2, wherein the lengths of the catheters vary.

4. The endoscope system of claim 3, wherein the stiffness levels of the catheters vary.

5. The endoscope system of claim 2, wherein the catheters are single-use catheters.

6. The endoscope system of claim 2, wherein each catheter includes a camera module.

7. The endoscope system of claim 6, wherein one of the camera modules is a disposable camera module designed for examining a patient's ear and another of the camera modules is a disposable camera module designed for examining a patient's nasal cavities.

8. The endoscope system of claim 6, wherein the image sensor sizes and optical characteristics of the camera modules vary.

9. The endoscope system of claim 6, wherein each catheter has a proximal end and a distal end, and has a connector at the proximal end.

10. The endoscope system of claim 9, wherein the connector has electrical contacts for relaying electrical and communication signals.

11. The endoscope system of claim 1, wherein the camera module includes an LED and a light pipe for transmitting light generated by the LED.

12. The endoscope system of claim 1, wherein the wall mounted unit includes a handle that is detachably connectable to the catheter.

13. The endoscope system of claim 12, wherein the wall mounted unit further includes a back panel, an interface module, an air pump that sends air to the handle.

14. The endoscope system of claim 1, wherein the LCD screen is a touch sensitive display having software controlled buttons, whereby an operator is able to perform control functions by touching the buttons.

15. An endoscope system comprising:
a flexiable catheter having a camera module, wherein the flexible catheter has no lumens and is not steerable; and
a control box that processes video images captured by the camera module and output video signals to a screen to display the captured video images.

16. A catheter comprising:
a tubular member; and
a camera module connected to an end of the tubular member, wherein the camera module has a diameter that is greater than a diameter of the tubular member.

17. The catheter of claim 16, wherein the diameter of the tubular member is 90%, 80%, 70%, 60%, 50%, 40% or 30% of the diameter of the camera module.

18. A method of detecting Barrett's esophagus, comprising
inserting a catheter of a gastroscope system into a patient's esophagus;
identifying an area of known esophageal tissue on a screen of the gastroscope system, and setting a first base line point in terms of image properties in the area of esophageal tissue;
identifying an area of known stomach epithelial tissue on the screen of the gastroscope system, and setting a second base line point in terms of image properties in the area of stomach epithelial tissue;
identifying areas of stomach epithelial cells on the screen based on the first and second base points; and
accentuating the identified areas of stomach epithelial cells.

19. The method of claim 18, wherein the step of identifying the areas of stomach epithelial cells includes analyzing the areas for various color properties.

20. The method of claim 18, further comprising measuring the degree of metaplasia by analyzing color properties.

21. A method of detecting Barrett's esophagus, comprising
inserting a catheter of a gastroscope system into a patient's esophagus;
identifying an area of known esophageal tissue on a screen of the gastroscope system, and setting a base line point in terms of image properties in the area of esophageal tissue;
identifying areas of stomach epithelial cells on the screen based on the base points; and
accentuating the identified areas of stomach epithelial cells.

22. A method of detecting Barrett's esophagus, comprising
inserting a catheter of a gastroscope system into a patient's esophagus;
identifying an area of known stomach epithelial tissue on the screen of the gastroscope system, and setting a base line point in terms of image properties in the area of stomach epithelial tissue;
identifying areas of stomach epithelial cells on the screen based on the base points; and
accentuating the identified areas of stomach epithelial cells.

23. A method for determining a length of metaplasia, the method comprising:
inserting a catheter of a gastroscope system into a patient's esophagus;
identifying upper and lower borders of the area of metaplasia;
moving a camera module of the gastroscope system from one of the upper and lower borders to the other while capturing partial images of the interior surface of the esophagus;
identifying similar regions or corresponding key points between two captured images;
calculating a distance by which a key point or corresponding area has moved from the earlier one of the two images to the later of the two images; and
obtaining a length of metaplasia by adding the calculated distances.

24. A method for determining an abnormal tissue, the method comprising:
setting a baseline level in a region of an esophagus to calibrate based on one or more factors; and
emphasizing regions of the esophagus that are dissimilar to the calibrated region.
